(19) United States
(12) Patent Application Publication
(10) Pub. No.: US 2017/0189318 A1
(43) Pub. Date: Jul. 6, 2017

(54) Skin, Nail and Hair Topical Antimicrobial Methods Using Formulations Containing Organosilane Quaternaries

(71) Applicant: Thomas L. Higgins, Lattingtown, NY (US)

(72) Inventor: Thomas L. Higgins, Lattingtown, NY (US)

(21) Appl. No.: 13/632,910

(22) Filed: Oct. 1, 2012

Related U.S. Application Data
(63) Continuation of application No. PCT/US2011/030697, filed on Mar. 31, 2011.
(60) Provisional application No. 61/319,596, filed on Mar. 31, 2010.

(51) Int. Cl. A61K 8/898; A01N 33/12; A61Q 17/00; A61K 8/41; A61K 9/00

(52) U.S. Cl. CPC A61K 8/898; A61K 8/416; A61K 9/0014; A61Q 17/005; A01N 33/12; A61K 31/135

(57) ABSTRACT

A method for treating human or animal skin, hands, feet, hair or nails to eliminate or reduce the number of microorganisms thereon by topical application of an aqueous formulation comprising antimicrobial organosilane quaternary ammonium compounds and benzalkonium chloride of the quaternary group, which provides antimicrobial and antiseptic activity. The method prevents reinfection for at least 12, preferably 24 and most preferably at least 60 hours. The formulation may further comprise methyl anthranilate, which performs the dual functions of fragrance (grape fragrance) and biocidal agent. The formulation may further comprise other compatible inert but desirable ingredients such as moisturizers, color and fragrance.

### CROSS REFERENCE TO RELATED APPLICATIONS

This application is a continuation of prior International Application No. PCT/US11/030697, filed Mar. 31, 2011, which claims the benefit of U.S. Provisional Application 61/319,596 filed Mar. 31, 2010.

### STATEMENT REGARDING FEDERALLY SPONSORED RESEARCH OR DEVELOPMENT

Not applicable.

### TECHNICAL FIELD OF INVENTION

This invention relates to the field of skin, hand, foot, nail and hair sanitizing compositions and methods.

### BACKGROUND OF THE INVENTION

Organosilanes containing quaternary ammonium halides and hydrolyzable alkoxy groups bonded to silicon have been employed in a wide variety of applications. The hydrolyzable groups enable these compounds to form bonds to substrates that contain hydroxy, alkoxy, oxide and similar reactive moieties. Organosilanes have been used to waterproof masonry and brick surfaces, as paint additives, fabric treatments and for surface modification of substrates that enhance miscibility in organic solvents or enable subsequent operations to be conducted on the substrate such as dyeing or painting.

Organosilane quaternary nitrogen compounds have also been employed effectively in eliminating and reducing bacterial, viral and fungal contamination when applied to a variety of surfaces including metal, glass, plastics, rubber, ceramics and fabrics including cellulose, cotton, acetates and nylon.

### DETAILED DESCRIPTION

An extended shelf-life, water-stable formulation of organosilane and benzalkonium chloride suitable for application to the hair, skin, hands, feet or nails of a human or animal, and methods for using the same are herein disclosed.

Upon application, the active portions of the organosilane and benzalkonium chloride are operative for reducing or eliminating microorganisms that may be present on the area to which the formulation is applied. The formulation is essentially non-toxic, non-flammable, uniformly dispersible, and simple and economical to use.

The formulation bonds to the skin, hair or nails and resists removal by washing or rinsing and provides broad spectrum antimicrobial activity.

The formulation of the invention comprises solubilized quaternary ammonium organosilanes. Most preferably, 3-(trihydroxysilyl) propyloctadecyldimethyl ammonium chloride is employed in the formulation. It is a water stable organosilane composition for treating various substrates and articles; the method for preparation of said compound is described in U.S. Pat. Nos. 5,959,014, 6,221,944 and 6,632,805, which are herein incorporated by reference as if fully set forth in their entireties. The formulation may be used as a component of a product adapted for topical application to the skin of a human or animal, such as, for example, antimicrobial and antiseptic hand sanitizers, foot remediators, antimicrobial skin cleansers, liquid antimicrobial hand soaps and antimicrobial skin moisturizing lotions and deodorant applications. Preferably, the formulation of the invention imparts antimicrobial, antiseptic, biocidal and moisturizing functionality to the skin. The functionality is of long duration, and preferably continues throughout normal daily activities for at least about twelve hours, preferably at least twenty-four hours, and most preferably at least about 60 hours, or more.

Other organosilane quaternary ammonium compounds may be used. Thus suitable organosilanes are selected from the group consisting of 3-(trihydroxysilyl)propyloctadecyldimethyl ammonium chloride, 3-(trihydroxysilyl)propyldidecylmethyl ammonium chloride, trisilanol derivatives, polysiloxanol derivatives and mixtures thereof

In another embodiment, the formulation of the invention comprises quaternary ammonium organosilicon compounds, the silanol derivatives, and mixtures thereof, such as 3-(trimethoxysilyl)-propyldimethyltetradecyl ammonium chloride.

The formulation may be made with or without nonionic surfactants such as ethoxylated alcohols; ethoxylated nonyl phenol(s); and ethoxylated alkyl phenol(s), 3-(trimethoxysilyl)propyldimethylhexadecyl ammonium chloride, 3-(dimethoxymethylsilyl) propyldimethyloctadecyl ammonium chloride and 3-(methoxydimethylsilyl)propyldimethyloctadecyl ammonium chloride.

Other suitable water stable organosilane compounds are described in U.S. Pat. Nos. 5,959,014, 6,221,944 and 6,632,805 which are herein incorporated by reference as if fully set forth in their entireties herein.

The formulation optionally, but preferably, also includes an ester of anthranilic acid as a biocidal and fragrance agent. A preferred fragrance agent, methyl anthranilate (C₈H₉NO₂), has a mild grape fragrance. Methyl anthranilate, also known as MA, methyl 2-aminobenzoate or carbomethoxyaniline, is an ester of anthranilic acid.

The preferred quaternary ammonium organosilanes containing hydrolyzable groups can form antimicrobially active, clear solutions in aqueous media which are stable over extended periods of time, which do not require addition of immiscible materials to form antimicrobially effective emulsions, and also do not require adjustment of pH. The compositions of the present invention can be readily and easily prepared by adding the quaternary ammonium organosilane composition to water, most preferably purified WFI (Water for Injection) grade water.

The addition of benzalkonium chloride provides additional antiseptic, bacteriostatic and fungistatic properties.

Most preferably, propylene glycol in an amount from about 0.1% to 20%, preferably between about 9%-11%, will be added to the formulation. This may provide additional beneficial effects, such as lowering the freezing point of the formulation, increasing the evaporation rate of the aqueous media from the skin, increasing the rate of antimicrobial effect, enhancing the cleansing properties, and/or increasing the solubility of other additives. Propylene glycol in this amount does not affect the extended stability or performance of the formulation.

Preferably, a carrier component suitable for topical application to the skin is employed in the formulation. Preferably, the formulation comprises from about 0.01 to 4.0 & w/v benzalkonium chloride ingredient and 0.5% to 2.5% w/v 3-(trihydroxysilyl)propyldimethyloctadecyl ammonium chloride and a (q.s) carrier component. Carrier components may be aqueous solutions or non-alcoholic water solutions, foams, fluids, creams or gels.

The formulation is homogeneous, may be scented and/or colored, and may contain water-soluble additives to enhance performance, biocidal activity and viscosity. One such preferred additive is methyl anthranilate. One of the characteristics of methyl anthranilate is that it provides the unique dual functionality of having biocidal properties, while also possessing a mild grape fragrance. One example of this biocidal property may be seen with Burkholderia cepacia, a bacterium which is so hardy that it has been found to persist even in the common topical antiseptic betadine. Burkholderia cepacia complex (BCC), or simply Burkholderia cepacia is a group of catalase-producing, non-lactose-fermenting Gram-negative bacteria composed of at least nine different species, including B. cepacia, B. multivorans, B. cenocepacia, B. vietnamiensis, B. stabilis, B. ambifaria, B. dolosa, B. anthina, and B. pyrrocinia. B. cepacia is an important human pathogen which most often causes pneumonia in immunocompromised individuals with underlying lung disease, such as cystic fibrosis or chronic granulomatous disease. Recently, a 0.2% chlorhexidine mouthwash was recalled after it was found to be contaminated with B. cepacia. The addition of methyl anthranilate prevents the growth of B. cepacia, as well as other bacteria that are known to survive in plastic containers.

The formulation of the invention can be applied to human skin and animal skin topically. When applied to the skin, the quaternary ammonium organosilane species forms an invisible (to the naked eye) macropolymeric polyionic layer on the skin that resists removal by repeated hand-washing or rinsing. Further, by inclusion of benzalkonium chloride in the formulation, the layer retains its antiseptic, antimicrobial and biocidal activity against a broad range of pathogens, and will remain active on the skin for at least about 12 hours, preferably at least about 24 hours, and most preferably at least about 60 hours. Most preferably, the formulation also retards moisture loss from the skin.

On application to the skin, the organosilane quaternary ammonium compounds form a water insoluble molecular layer, which is substantive to the skin and will remain through a minimum of nine rinses. Skin treated with the above described materials, inclusive of benzalkonium chloride, are resistant to infection from gram-positive and gram-negative bacteria, viruses, and fungi, including mold and spores. Application of these formulations promotes a smooth feeling to the skin and protects against redness and chapping due to the moisturizing qualities of the material.

The compositions of the present invention are non-toxic, efficient and economical for use as topical antimicrobial and antiseptic skin-treatments. Conventional non-silicon containing quaternary ammonium compounds, when applied to the skin, are easily removed by rinsing or hand washing. They exhibit no substantive characteristics on skin. Following application, organosilane quaternary ammonium compounds comprising benzalkonium chloride are substantive to the skin and exhibit antimicrobial, antiseptic and biocidal activity while on the skin, reducing or eliminating existing pathogens, and preventing re-infection by reducing or eliminating additional pathogens introduced after application. These compounds do not demonstrate irritability to the skin presumably due to their unique bonding abilities which effectively places a barrier of organosiloxy moieties between the skin surface and the antimicrobial active ammonium cation. These antimicrobial barriers prevent re-infection on subsequent introduction of pathogens and function as a moisturizing layer by slowing moisture loss from the surface of the skin.

Accordingly, in one embodiment, this invention provides a method of treating human or animal skin and/or nails to reduce or eliminate microorganisms on said skin, comprising topical application of a formulation comprising a water soluble quaternary ammonium organosilane and benzalkonium chloride, for a period of time sufficient to render the substrate antimicrobially and antiseptically active. Most preferably, the formulation further comprises additives, one of which is methyl anthranilate that functions as a biocide and a fragrance, and the formulation further comprises other additives and thickeners which when applied to the skin will cleanse the skin of dirt and oils. The formulation may optionally include colors or scents which do not interfere with the antimicrobial activity.

A method for rendering skin, nails and hair antimicrobial, comprising topical application thereto of a quaternary ammonium organosilane, said formulation containing benzalkonium chloride of the quaternary ammonium group, which adheres to the skin, hair and nails, retains its antimicrobial and antiseptic activity for at least about 12, preferably at least about 24, or most preferably at least about 60 hours and prevents re-infection of the skin, hair and nails on subsequent contact with infection producing pathogens is provided.

In another embodiment, this invention provides a method for preparing a clear water-stable composition comprising the quaternary ammonium organosilane of this invention, benzalkonium chloride and water, or a water and a no or low alcohol solution.

In the Examples that follow, the formulation can be made with and without methyl anthranilate. If made without, an equivalent percentage of water can be used.

### EXAMPLE 1

Sample Formulation: The following formulation was prepared for use as a skin sanitizing composition.

| | |
|---|---|
| 3-(Trihydroxysilyl)propyldimethy- | <1.0% |
| loctadecyl ammonium chloride | |
| Benzalkonium Chloride | 0.10% |
| Propylene glycol | <10% |
| Methyl Anthranilate (fragrance) | <0.3% |
| Purified water | <92% |

### SECOND EXAMPLE OF FORMULA

| | |
|---|---|
| 3-(Trihydroxysilyl)propyl- | <1.0% |
| dimethyloctadecyl | |
| Benzalkonium Chloride | 0.10% |
| Propylene glycol | <10% |
| Purified water | <91% |
| Ethoxylated alcohol | <3% |

Test parameters for Skin Sanitizer: The following parameters were used for the testing the sample formulation.
Size of the slide: 1×1 inch
Original inoculum Conc.: 10⁸ (Mac #1) [Macrophage-1 antigen (or integrin alphaMbeta2) is a complement receptor]
Inoculum used: 0.05 ml
Amount of Sanitizer used: 1 spray
Amount of Tween 80 used: 0.1 mL
Organism used: MRSA-BAA 44
Amount of Diluent used (DE Neutralizer Broth): 5 mL
Contact time: 1 hr, 3 hr, 6 hr, 8 hr & 24 hr
Shaker time: 5 min.
Drying time in Incubator: 40 min.
Replicates: 5
Serial dilution: For control: 10¹

Testing Procedure for Skin Sanitizer: The following procedure was used for testing the sample formulation.
1. Prepared 10 mL of the inoculum of Mac #1 from a 24 hr stock culture.
2. Aseptically added 0.05 mL inoculum to the sterile 1×1 inch slide and spread evenly.
3. Let air dry in the incubator at 35° C. for 40 min.
4. After air dry, sprayed the slides with 1 spray (approximately 0.75 mL) of the sanitizer.
5. Let the slides sit for the contact times of 1 hr, 3 hr, 6 hr, 8 hr and 24 hr.
6. After each contact time, transferred the slides aseptically into sterile specimen cup and added 5 mL of the neutralizer broth (DE broth).
7. Agitated specimen cup for 5 min. on the shaker.
8. Sub-cultured onto the plates at various dilutions. For control, serial dilution done.
9. The counts and dilution were recorded at 24 hrs.

TABLE 1
Results for Skin Sanitizer:

| | | Sanitizer | | | Control | | |
|---|---|---|---|---|---|---|---|
| Contact | Sam- | Raw | Dilution | Final | Raw | Dilution | Final |
| time | ple | count | read | count | count | read | count |
| 1 HR | 1 | NG | | NG | 128 | 0.001 | 6.4 × 10⁶ |
| | 2 | 19 | 0.01 | 9.5 × 10³ | 116 | 0.001 | 5.8 × 10⁶ |
| | 3 | 2 | 0.001 | 1.0 × 10⁴ | 160 | 0.001 | 8.0 × 10⁶ |
| | 4 | 11 | 0.001 | 5.5 × 10⁴ | 108 | 0.001 | 5.4 × 10⁶ |
| | 5 | 25 | 0.001 | 1.2 × 10⁵ | 76 | 0.001 | 3.8 × 10⁶ |
| 3 HR | 1 | 53 | 0.001 | 2.6 × 10⁵ | 168 | 0.001 | 8.4 × 10⁶ |
| | 2 | 22 | 0.001 | 1.1 × 10⁵ | 144 | 0.001 | 7.2 × 10⁶ |
| | 3 | 7 | 0.001 | 3.5 × 10⁴ | 132 | 0.001 | 6.6 × 10⁶ |
| | 4 | 25 | 0.001 | 1.2 × 10⁵ | 112 | 0.001 | 5.6 × 10⁶ |
| | 5 | 40 | 0.001 | 2.0 × 10⁵ | 236 | 0.001 | 1.1 × 10⁷ |
| 6 HR | 1 | 56 | 0.001 | 2.8 × 10⁵ | 192 | 0.001 | 9.6 × 10⁶ |
| | 2 | 52 | 0.001 | 2.6 × 10⁵ | 168 | 0.001 | 8.4 × 10⁶ |
| | 3 | 50 | 0.001 | 2.5 × 10⁵ | 168 | 0.001 | 8.4 × 10⁶ |
| | 4 | 6 | 0.001 | 3.0 × 10⁴ | 184 | 0.001 | 9.2 × 10⁶ |
| | 5 | 46 | 0.001 | 2.3 × 10⁵ | 176 | 0.001 | 8.8 × 10⁶ |
| 8 HR | 1 | 42 | 0.001 | 2.1 × 10⁵ | 152 | 0.001 | 7.6 × 10⁶ |
| | 2 | 2 | 0.001 | 1.0 × 10⁴ | 172 | 0.001 | 8.6 × 10⁶ |
| | 3 | 3 | 0.001 | 1.5 × 10⁴ | 132 | 0.001 | 6.6 × 10⁶ |
| | 4 | 8 | 0.01 | 4.0 × 10³ | 172 | 0.001 | 8.6 × 10⁶ |
| | 5 | 34 | 0.001 | 1.7 × 10⁵ | 132 | 0.001 | 6.6 × 10⁶ |
| 24 HR | 1 | 30 | 0.001 | 1.5 × 10⁵ | 154 | 0.001 | 7.7 × 10⁶ |
| | 2 | 3 | 0.001 | 1.5 × 10⁴ | 224 | 0.001 | 1.1 × 10⁷ |
| | 3 | 11 | 0.001 | 5.5 × 10⁴ | 157 | 0.001 | 7.8 × 10⁶ |
| | 4 | 33 | 0.001 | 1.6 × 10⁵ | 180 | 0.001 | 9.0 × 10⁶ |
| | 5 | 8 | 0.01 | 4.0 × 10³ | 128 | 0.001 | 6.4 × 10⁶ |

Calculation:
For Challenge: Raw count × dilution read × Amount of Diluent (DE broth) 5 ml
For Control: Raw count × Dilution read × Amount of Diluent (DE broth) 5 ml × Serial dilution (10\hat{ }1)

### EXAMPLE 2

### Virucidal Efficacy of Hand Rinse Formulation

Protocol: The following protocol was followed for testing the formulation on inanimate environmental surfaces, and the use can be extended to hair, skin, nails and other natural surfaces.
1) ASTM E1053-97 (2002) Standard Test Method for Efficacy of Virucidal Agents Intended for Inanimate Environmental Surfaces,
2) ASTM E1482-04 Standard Test Method for Neutralization of Virucidal Agents in Virucidal Efficacy Evaluations
3) U.S. E.P.A. Pesticide Assessment Guidelines, Subdivision G: Product Performance, Section 91-2 (f), and Section 91-30, (d),

TABLE 2
Virucidal efficacy test of Hand Sanitizer Formulation

| | | Virus | | | |
|---|---|---|---|---|---|
| | | dry | | | |
| | | film + | Virus | Neutralization | Neutralization |
| | | column | film + | infectivity | cytotoxicity |
| Dilution | Virus titer | control | GS5 | control | control |
| 10⁻¹ | ++++ | ++++ | −−−− | ++++ | −−−− |
| 10⁻² | ++++ | ++++ | −−−− | ++++ | −−−− |
| 10⁻³ | ++++ | +++− | −−−− | ++++ | −−−− |
| 10⁻⁴ | ++++ | −−−− | −−−− | ++++ | −−−− |
| 10⁻⁵ | +−−− | −−−− | −−−− | ++++ | −−−− |
| 10⁻⁶ | −−−− | −−−− | −−−− | ++++ | −−−− |
| 10⁻⁷ | −−−− | −−−− | −−−− | ++++ | −−−− |
| TCID₅₀/ | 10^{4.66} | 10^{3.33} | ≦10^{0.5} | | |
| 0.1 mL | | | | | |

+ virus-specific cytopathic effect observed;
− no virus-specific cytopathic effect or cytotoxicity observed.

Virus and Cell Cultures: The following cultures were used for testing the formulation on inanimate environmental surfaces.

MDCK Cell Culture: Madin Darby Canine Kidney (MDCK) ATCC CCL-34 was obtained from the American Type Culture Collection. MDCK cells were cultured in DMEM Dulbecco's Minimum Essential Medium (DMEM) with 2.50 mM L-Glutamine, 15 mM HEPES Buffer, and 10% fetal bovine serum (FBS). The MDCK cultures were incubated under 5% CO₂ at 37° C. for 24˜48 hours to form monolayers before testing.

Virus Culture: Influenza A virus (H1N1) ATCC VR-1469 was obtained from the American Type Culture Collection. Typically, monolayer MDCK cultures were washed twice with PBS buffer. Then, 10˜15% VR-1469 were inoculated and incubated at 37° C. for 1 hour. At intervals of 15 min. shaking the flask is done to allow the virus attach to MDCK cells. After 1 hour, medium was removed and culture washed with PBS. Maintenance medium (DMEM with 2 μg/mL TPCK-trypsin without FBS) was added. Culture was returned to 37° C. incubator for 48˜72 hrs. (or see the CPE, if the CPE comes out more than 90%), and then the virus was harvested. The virus stock culture was stored at −80° C. freezer until testing.

Calculations of TCID50: Viral titers are expressed as 50% titration end point infectivity (TCID₅₀) by the Reed-Muench method. Proportional distance formula =[(% positive value>50%)−50%]/[(% positive value<50%)]. Log infectious dose 50=(log dilution>50%)+(proportional distance×dilution factor).

Testing Procedure: The following procedure was followed for testing the formulation on inanimate environmental surfaces.

Neutralization Control Test: A small wad of glass wool was placed in a 5-cc syringe. Sephacryl S-1000 SF was added to the syringe to form a 3-cc column volume. The column was calibrated with 10 mL PBS and then centrifuged at 600×g for 3 min. Next, 0.6 mL GS solution was pipetted onto the column. The column was placed in a 15 mL conical centrifuge tube and centrifuged for 3 min at 600×g. A set of 10-fold dilutions was prepared of the filtrate and 50 μL amounts were added to monolayer MDCK cultures on 96-well plates. Plates were incubated for 1 hr and had 100 μL DMEM medium added. Plates were incubated at 37° C. and cell growth observed (neutralization cytotoxicity control). Additionally, each 10-fold dilution of the filtrate was mixed with 2 log₁₀ virus, and the mixture placed into monolayer MDCK cultures on 96-well plates to observe viral infectivity (neutralization infectivity control).

Virus Film Control: 0.2 mL of virus was spread on 60 mm Petri plates and virus film allowed to dry for 1 hour at room Temperature. Next, 2 mL PBS was added over the virus film and allowed to stand for 10 min. Then the surface was scraped with sterile rubber policeman to suspend virus and 0.6 mL amounts were pipetted onto the Sephacryl S-1000 column. After centrifugation of the column at 600×g for 3 min, serial 10-fold dilutions of the filtrate were prepared and 50 μL of each dilution were added onto the monolayer MDCK cultures and one hour at 37° C. was allowed for virus absorption. Then 100 μL virus maintenance medium was added. After incubation at 37° C., cell growth was observed for evidence of cytopathic effect.

Virucidal Test: On each virus film, 2 mL use dilution of GS was added and allowed to stand for 10 min. at room temperature. Petri plate surfaces were scraped to suspend virus, 0.6 mL virus/GS mixture was pipetted onto the column, and the column centrifuged at 600×g for 3 min. Serial 10-fold dilutions of the filtrate were prepared, and 50 μL of each dilution added onto the monolayer MDCK cultures. Cultures were allowed to stand one hour at 37° C. and then 100 μL virus maintenance medium was added. Cultures were incubated at 37° C. and cell growth observed for evidence of cytopathic effect.

Test Results: The following Table 2 shows results of a foam comprising:

| | |
|---|---|
| Benzalkonium chloride | 0.1% |
| Propylene glycol | 9.0% |
| 3 trihydroxysilylpropyldimethyl- | 0.9% |
| octadecyl ammonium chloride | |
| Purified Water | 90.0% |

exposed to Influenza A virus (H1N1) ATCC VR-1469 on inanimate surface. The titer of virus control and virus dry film control were 4.66 and 3.33 log₁₀ respectively. In neutralization cytotoxicity control test, the filtrate didn't show cytotoxicity against MDCK cells. After the virus film exposure to the foam, the viral titer was tested as ≦10^{0.5}, and the reduction of influenza A virus VR-1469 was ≧2.83 log₁₀.

TABLE 2
Virucidal efficacy test of Hand Sanitizer Formulation

| | | Virus | | | |
|---|---|---|---|---|---|
| | | dry | | | |
| | | film + | Virus | Neutralization | Neutralization |
| | | column | film + | infectivity | cytotoxicity |
| Dilution | Virus titer | control | GS5 | control | control |
| 10⁻¹ | ++++ | ++++ | −−−− | ++++ | −−−− |
| 10⁻² | ++++ | ++++ | −−−− | ++++ | −−−− |
| 10⁻³ | ++++ | +++− | −−−− | ++++ | −−−− |
| 10⁻⁴ | ++++ | −−−− | −−−− | ++++ | −−−− |
| 10⁻⁵ | +−−− | −−−− | −−−− | ++++ | −−−− |
| 10⁻⁶ | −−−− | −−−− | −−−− | ++++ | −−−− |
| 10⁻⁷ | −−−− | −−−− | −−−− | ++++ | −−−− |
| TCID₅₀/ | 10^{4.66} | 10^{3.33} | ≦10^{0.5} | | |
| 0.1 mL | | | | | |

+ virus-specific cytopathic effect observed;
− no virus-specific cytopathic effect or cytotoxicity observed.

I claim:

1. A method for treating skin, nails and/or hair to reduce or eliminate microbial organisms and to prevent re-infection in the event of subsequent contact with additional microbial organisms for at least about 12 hours, comprising application of a formulation containing a quaternary ammonium organosilane and benzalkonium chloride.

2. The method of claim 1, wherein said formulation further comprises an ester of anthranilic acid.

3. The method of claim 2, wherein said ester is methyl anthranilate.

4. The method of claim 1, wherein said quaternary ammonium organosilane is selected from the group consisting of 3-(trihydroxysilyl)-propyloctadecyldimethyl ammonium chloride, 3-(trihydroxysilyl)-propyldidecylmethyl ammonium chloride, trisilanol derivatives, polysiloxanol derivatives and mixtures thereof.

5. The method of claim 1, wherein said formulation comprises a silanol derivative selected from the group consisting of 3-(trimethoxysilyl)-propyldimethyltetradecyl ammonium chloride, 3-(trimethoxysilyl)propyldimethylhexadecyl ammonium chloride, 3-(dimethoxymethylsilyl) propyldimethyloctadecyl ammonium chloride and 3-(methoxydimethylsilyl)propyldimethyloctadecyl ammonium chloride.

6. The method of claim 4, wherein said formulation further comprises a silanol derivative selected from the group consisting of 3-(trimethoxysilyl)propyldimethyltetradecyl ammonium chloride, 3-(trimethoxysilyl)propyldimethylhexadecyl ammonium chloride, 3-(dimethoxymethylsilyl) propyldimethyloctadecyl ammonium chloride and 3-(methoxydimethylsilyl)propyldimethyloctadecyl ammonium chloride.

7. The method of claim 1, wherein said formulation further comprises propylene glycol in an amount of 0.1 to 20% by weight of the total formulation.

8. The method of claim 1 in which said quaternary ammonium organosilane is present at a concentration of from about 0.01% to about 10% by weight.

9. The method of claim 1 in which said quaternary ammonium organosilane is 3-(trihydroxysilyl)-propyldimethyloctadecyl ammonium chloride.

10. The method of claim 1 wherein said formulation further comprises a carrier selected from a foam, gel, lotion, cream or soap.

11. The method of claim 1, wherein said formulation further comprises one or more water-soluble additives.

12. The method of claim 11, in which said water-soluble additive is methyl anthranilate in an amount from 0.005% to 10% of said formulation.

13. The method of claim 11, in which water-soluble additive is propylene glycol in an amount from about 9 to 11% of said formulation.

14. The method of claim 1, wherein the amount of said quaternary ammonium organosilane in said applied formulation is sufficient to maintain said reduction or elimination of pathogens for at least 60 hours.

15. The method of claim 1, wherein said quaternary ammonium organosilane constitutes 0.1 to 3 weight percent of said formulation.

16. The method of claim 1, wherein said formulation protects against reinfection for at least about 24 hours.

17. The method of claim 1, wherein said formulation protects against reinfection for at least about 60 hours.

18. The method of claim 1, wherein said formulation further comprises a non-ionic surfactant.

19. The method of claim 18, wherein said nonionic surfactant is selected from the group consisting of ethoxylated alcohols; ethoxylated nonyl phenol(s); and ethoxylated alkyl phenol(s).

20. A formulation for topical application to human or animal skin, hair or nails, comprising from about 0.01 to 4.0 percent w/w of a quaternary ammonium organosilane in an aqueous carrier and benzalkonium chloride ingredient in an amount from about 0.1% to 2.5% w/w.

21. The formulation of claim 20, wherein said formulation further comprises methyl anthranilate in an amount from about 0.005% to 10% w/w.

22. The formulation of claim 21, further comprising propylene glycol in an amount from about 0.1% to 20% w/w.

23. The formulation of claim 20, wherein said quaternary ammonium organosilane is selected from the group consisting of 3-(trihydroxysilyl)-propyloctadecyldimethyl ammonium chloride, 3-(trihydroxysilyl)-propyldidecylmethyl ammonium chloride, trisilanol derivatives, polysiloxanol derivatives and mixtures thereof.

24. The formulation of claim 20, wherein said quaternary ammonium organosilane is 3-(trihydroxysilyl)-propyldimethyloctadecyl ammonium chloride.

25. The formulation of claim 20, wherein said quaternary ammonium organosilane is selected from the group consisting of 3-(trimethoxysilyl)-propyldimethyltetradecyl ammonium chloride, 3-(trimethoxysilyl)propyldimethylhexadecyl ammonium chloride, 3-(dimethoxymethylsilyl) propyldimethyloctadecyl ammonium chloride and 3-(methoxydimethylsilyl)propyldimethyloctadecyl ammonium chloride.

26. The formulation of claim 23, further comprising a quaternary ammonium organosilane selected from the group consisting of 3-(trimethoxysilyl)propyldimethyltetradecyl ammonium chloride, 3-(trimethoxysilyl)propyldimethylhexadecyl ammonium chloride, 3-(dimethoxymethylsilyl) propyldimethyloctadecyl ammonium chloride and 3-(methoxydimethylsilyl)propyldimethyloctadecyl ammonium chloride.

27. The formulation of claim 24, further comprising a quaternary ammonium organosilane selected from the group consisting of 3-(trimethoxysilyl)propyldimethyltetradecyl ammonium chloride, 3-(trimethoxysilyl)propyldimethylhexadecyl ammonium chloride, 3-(dimethoxymethylsilyl) propyldimethyloctadecyl ammonium chloride and 3-(methoxydimethylsilyl)propyldimethyloctadecyl ammonium chloride.

28. The formulation of claim 20, further comprising a non-ionic surfactant.

29. The formulation of claim 28, wherein said non-ionic surfactant is selected from the group consisting of ethoxylated alcohols; ethoxylated nonyl phenol(s); and ethoxylated alkyl phenol(s).
